(19) United States
(12) Patent Application Publication
(10) Pub. No.: US 2020/0330485 A1
(43) Pub. Date: Oct. 22, 2020

(54) METHOD AND COMPOSITION FOR REVERSING AND/OR INHIBITING ATHEROSCLEROSIS

(71) Applicant: ARJIL BIOTECH HOLDING COMPANY LIMITED, Hsinchu (TW)

(72) Inventors: Yeh B Wu, Hsinchu City (TW); Jir-Mehng Lo, Hsinchu City (TW); Hui Ju Liang, Taipei City (TW); Pei-Hsin Lin, Hsinchu County (TW); Chieh-Hsi Wu, Taichung City (TW)

(73) Assignee: ARJIL BIOTECH HOLDING COMPANY LIMITED, Hsinchu (TW)

(21) Appl. No.: 16/851,637

(22) Filed: Apr. 17, 2020

Related U.S. Application Data
(60) Provisional application No. 62/835,663, filed on Apr. 18, 2019.

(51) Int. Cl. A61K 31/575; A61K 36/07; A61P 9/10

(52) U.S. Cl. CPC A61K 31/575; A61P 9/10; A61K 36/07

(57) ABSTRACT

The present invention pertains to a method for inhibiting, reducing or reversing atherosclerosis or a composition or pharmaceutical composition for preventing or treating an atherosclerotic disease containing a preparation of Antrodia camphorata, and/or one or more active compounds isolated form Antrodia camphorataa.

### CROSS REFERENCE TO RELATED APPLICATIONS

This application claims the benefit of and priority to U.S. Provisional Application Ser. No. 62/835,663, filed Apr. 18, 2019, which is incorporated herein by reference in their entirety.

### FIELD OF THE INVENTION

The present invention provides a method and composition of reversing and/or inhibiting atherosclerosis.

### BACKGROUND OF THE INVENTION

Atherosclerosis is a fundamental pathological process that is known to cause some serious cardiovascular diseases, including stroke and coronary artery disease. Although classical risk factors for atherosclerosis may include dyslipoproteinaemia, diabetes, cigarette smoking, hypertension and genetic abnormalities, hypercholesterolaemia is considered one of the main triggers of atherosclerosis. The increase in plasma cholesterol levels results in changes of the arterial endothelial permeability that allow the migration of lipids, especially low-density lipoprotein cholesterol (LDL-C) particles, into the arterial wall to form a plaque. When a plaque covers more than 40% of internal elastic layer of the vessel, the arterial channel is considered to be occupied, thereby obstructing the blood flow.

Currently, treatment for atherosclerosis include medications to lower cholesterol or to decrease clotting, and surgical management. Surgical therapies only treat isolated lesions, and plaques downstream from the treated lesion may continue to obstruct blood flow. Furthermore, surgical therapies are associated with the late complication of restenosis. On the other hand, medications to lower cholesterol such as statins reduce cardiovascular events by only about 20%-40%.

What is needed for atherosclerosis treatment not only slows progression of lesions, but also causes regression and shrinkage of established plaques.

### BRIEF SUMMARY OF THE INVENTION

It is unexpectedly found in the present invention that a preparation of Antrodia camphorata, and active ingredients of Antrodia camphorata are effective to reverse and/or inhibit atherosclerosis.

It was ascertained in the examples that the preparation of Antrodia camphorata was able to reverse and inhibit an aortic fatty streak lesion, a plaque formation and a vascular restenosis.

It is an object of the present invention to provide a method for preventing and/or treating atherosclerotic disease, comprising administering to a subject in need thereof a preparation of Antrodia camphorate and/or active ingredients of Antrodia camphorata.

Actually, it would be derived from the findings that the reversion or inhibition of atherosclerosis by inhibiting the aortic fatty streak lesion, the plaque formation, and the vascular restenosis to inhibit ischemic stroke, cardiovascular disease, peripheral arterial disease and major organ vascular atherosclerosis.

In another object, the present invention provides a pharmaceutical composition for preventing and/or treating atherosclerotic disease, which comprises a therapeutically effective amount of a preparation of Antrodia camphorate and/or active ingredients of Antrodia camphorata, and a pharmaceutically acceptable carrier.

In one embodiment of the invention, the preparation of Antrodia camphorate includes but not is limited to an extract of Antrodia camphorate, an extract of a dish culture of Antrodia camphorate, an extract of Antrodia camphorata fruit body, and active compounds isolated from the above-mentioned extracts.

In one example of the invention, the active compound may be one or more selected from the group consisting of:

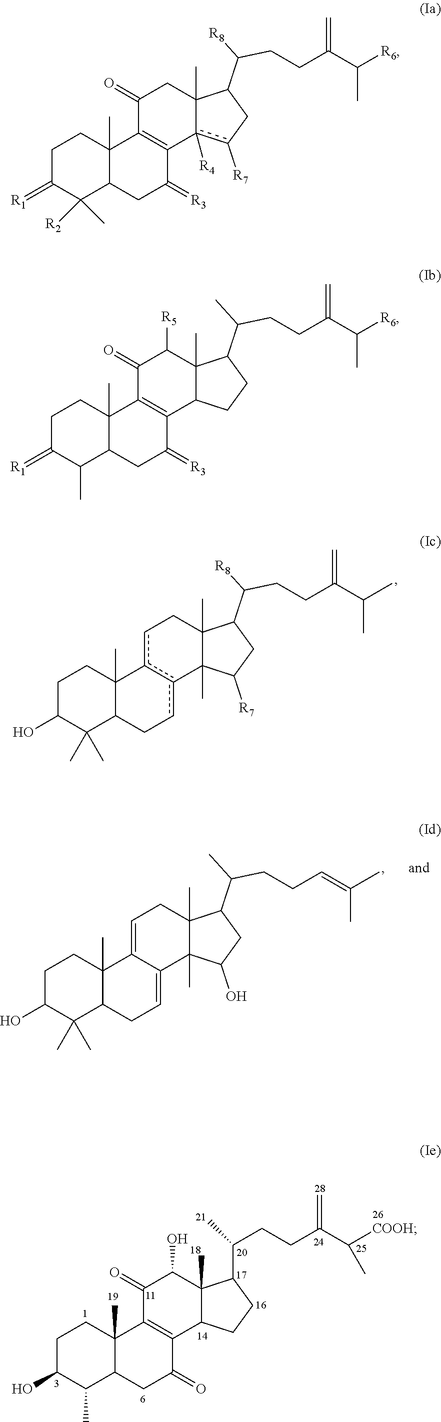

wherein R₁ is O, α-OH or β-H; R₂ is H or OH; R₃ is O, α-H, β-OAc or H₂; R₄ is H or OH; R₅ is H or OH; R₆ is COOH or COO(CH₂)n-CH₃; R₇ is H, OH, or OAc; R₈ is CH₃ or COOH; the dotted line represents a single bond or a double bond; n is an integer from 0-3.

In one particular example of the present invention, the compound is dehydroeburicoic acid:

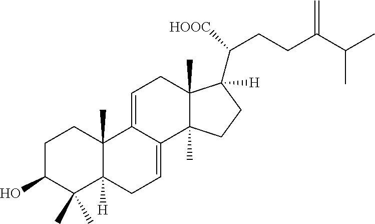

In another example of the present invention, the compound is dehydrosulphurenic acid (dehydrosulfurenic acid):

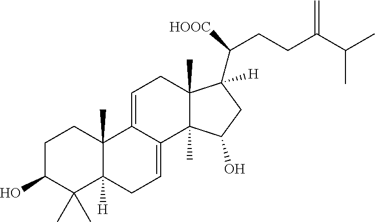 [SMILES: C=C(CC[C@H](C(=O)O)[C@H]1C[C@H](O)[C@@]2(C)C3=CC[C@H]4C(C)(C)[C@@H](O)CC[C@]4(C)C3=CC[C@]12C)C(C)C]

In one example of the present invention, the compound is antcin A:

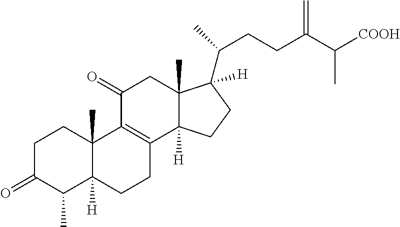 [SMILES: C=C(CC[C@@H](C)[C@H]1CC[C@H]2C3=C(C(=O)C[C@]12C)[C@@]1(C)CCC(=O)[C@@H](C)[C@@H]1CC3)C(C)C(=O)O]

In one example of the present invention, the compound is antcin B:

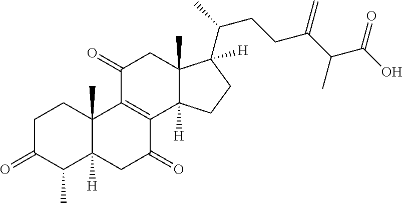 [SMILES: C=C(CC[C@@H](C)[C@H]1CC[C@H]2C3=C(C(=O)C[C@]12C)[C@@]1(C)CCC(=O)[C@@H](C)[C@@H]1CC3=O)C(C)C(=O)O]

In one example of the present invention, the compound is antcin C:

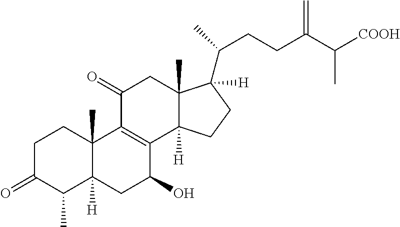 [SMILES: C=C(CC[C@@H](C)[C@H]1CC[C@H]2C3=C(C(=O)C[C@]12C)[C@@]1(C)CCC(=O)[C@@H](C)[C@@H]1C[C@@H]3O)C(C)C(=O)O]

In one example of the nresent invention, the comnound is antcin H:

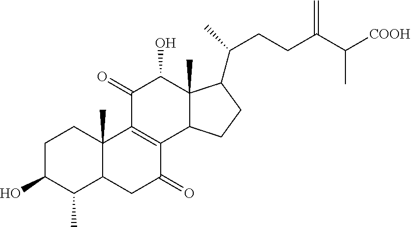 [SMILES: C=C(CC[C@@H](C)C1CCC2C3=C(C(=O)[C@H](O)[C@@]21C)[C@@]1(C)CC[C@H](O)[C@@H](C)C1CC3=O)C(C)C(=O)O]

In one example of the present invention, the compound is antcin K:

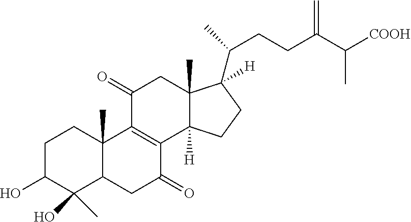 [SMILES: C=C(CC[C@@H](C)[C@H]1CC[C@H]2C3=C(C(=O)C[C@]12C)[C@@]1(C)CCC(O)[C@](C)(O)C1CC3=O)C(C)C(=O)O]

In one further aspect, the present invention provides a method for preventing or treating an atherosclerotic disease comprising administering to a subject in need thereof a therapeutically effective amount of a composition/pharmaceutical composition as disclosed herein, and at least one additional atherosclerosis therapeutic agent.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows changes of body weight. ND, standard rabbit chow; HF, standard rabbit chow containing 0.5% cholesterol; L, standard rabbit chow containing both 0.5% cholesterol and 10 mg/kg Lovastatin; ARH003, standard rabbit chow containing both 0.5% cholesterol and 1% ARH003; ARH004, standard rabbit chow containing both 0.5% cholesterol and 1% ARH004; P, standard rabbit chow containing both 0.5% cholesterol and 10 mg/kg Pure compound.

FIG. 2 shows levels of triglyceride (left panel) and total cholesterol (right panel) at week 0. ND, standard rabbit chow; HF, standard rabbit chow containing 0.5% cholesterol; L, standard rabbit chow containing both 0.5% cholesterol and 10 mg/kg Lovastatin; ARH003, standard rabbit chow containing both 0.5% cholesterol and 1% ARH003; ARH004, standard rabbit chow containing both 0.5% cholesterol and 1% ARH004; P, standard rabbit chow containing both 0.5% cholesterol and 10 mg/kg Pure compound.

FIG. 3 shows levels of triglyceride (left panel) and total cholesterol (right panel) at week 12. ND, standard rabbit chow; HF, standard rabbit chow containing 0.5% cholesterol; L, standard rabbit chow containing both 0.5% cholesterol and 10 mg/kg Lovastatin; ARH003, standard rabbit chow containing both 0.5% cholesterol and 1% ARH003; ARH004, standard rabbit chow containing both 0.5% cholesterol and 1% ARH004; P, standard rabbit chow containing both 0.5% cholesterol and 10 mg/kg Pure compound. t and * indicate a P<0.05 as compared with the control group and HF group, respectively.

FIG. 4 shows a histopathochemical examination of aortic fatty streak lesions. ND, standard rabbit chow; HF, standard rabbit chow containing 0.5% cholesterol; L, standard rabbit chow containing both 0.5% cholesterol and 10 mg/kg Lovastatin; ARH003, standard rabbit chow containing both 0.5% cholesterol and 1% ARH003; ARH004, standard rabbit chow containing both 0.5% cholesterol and 1% ARH004; P, standard rabbit chow containing both 0.5% cholesterol and 10 mg/kg Pure compound.

FIG. 5 shows a HE staining of coronary artery sections. ND, standard rabbit chow; HF, standard rabbit chow containing 0.5% cholesterol; Lovastatin, standard rabbit chow containing both 0.5% cholesterol and 10 mg/kg Lovastatin; ARH003, standard rabbit chow containing both 0.5% cholesterol and 1% ARH003; ARH004, standard rabbit chow containing both 0.5% cholesterol and 1% ARH004; Pure compound, standard rabbit chow containing both 0.5% cholesterol and 10 mg/kg Pure compound.

FIG. 6 shows a HE staining of coronary artery sections magnified. ND, standard rabbit chow; HF, standard rabbit chow containing 0.5% cholesterol; Lovastatin, standard rabbit chow containing both 0.5% cholesterol and 10 mg/kg Lovastatin; ARH003, standard rabbit chow containing both 0.5% cholesterol and 1% ARH003; ARH004, standard rabbit chow containing both 0.5% cholesterol and 1% ARH004; Pure compound, standard rabbit chow containing both 0.5% cholesterol and 10 mg/kg Pure compound; N, neointima layer; M, media layer.

FIG. 7 shows a manifestation of vascular restenosis presented as the ratio of neointima-to-media area. ND, standard rabbit chow; HF, standard rabbit chow containing 0.5% cholesterol; Lova, standard rabbit chow containing both 0.5% cholesterol and 10 mg/kg Lovastatin; ARH003, standard rabbit chow containing both 0.5% cholesterol and 1% ARH003; ARH004, standard rabbit chow containing both 0.5% cholesterol and 1% ARH004; P, standard rabbit chow containing both 0.5% cholesterol and 10 mg/kg Pure compound. * P<0.05; **, P<0.01.

FIG. 8 shows a histopathochemical examination of liver tissues in the hypercholesterolemic rabbit model after the 12-week study. ND, standard rabbit chow; HF, standard rabbit chow containing 0.5% cholesterol; Lovastatin, standard rabbit chow containing both 0.5% cholesterol and 10 mg/kg Lovastatin; ARH003, standard rabbit chow containing both 0.5% cholesterol and 1% ARH003; ARH004, standard rabbit chow containing both 0.5% cholesterol and 1% ARH004; Pure compound, standard rabbit chow containing both 0.5% cholesterol and 10 mg/kg Pure compound.

### DETAILED DESCRIPTION OF THE INVENTION

The above summary of the present invention will be further described with reference to the embodiments of the following examples. However, it should not be understood that the content of the present invention is only limited to the following embodiments, and all the inventions based on the above-mentioned contents of the present invention belong to the scope of the present invention.

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

The present invention provides a method for preventing and/or treating atherosclerotic disease, comprising administering to a subject in need thereof a preparation of Antrodia camphorate and/or active ingredients of Antrodia camphorata.

The present invention also provides a compositon/pharmaceutical composition for preventing and/or treating atherosclerotic disease, which comprises a therapeutically effective amount of a preparation of Antrodia camphorate and/or active ingredients of Antrodia camphorata, and a pharmaceutically acceptable carrier.

According to the invention, the preparation of Antrodia camphorate includes but not is limited to an extract of Antrodia camphorate, an extract of a dish culture of Antrodia camphorate, an extract of Antrodia camphorata fruit body, and active compounds isolated from the above-mentioned extracts, and the derivatives thereof.

More particularly, the active compounds isolated from Antrodia camphorata are one or more selected from the group consisting of the following:

(1)

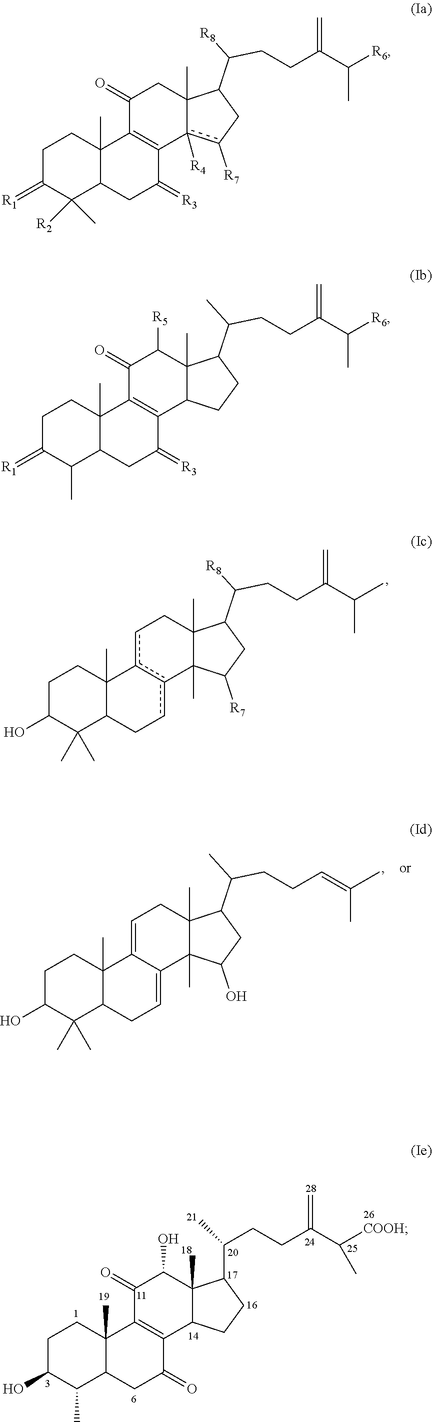

wherein R₁ is O, α-OH or β-H; R₂ is H or OH; R₃ is O, α-H, β-OAc or H₂; R₄ is H or OH; R₅ is H or OH; R₆ is COOH or COO(CH₂)n-CH₃; R₇ is H, OH, or OAc; R₈ is CH₃ or COOH; the dotted line represents a single bond or a double bond; n is an integer from 0-3.

In the example of the invention, the compound may be:

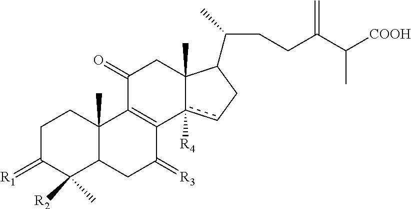

| | R₁ | R₂ | R₃ | R₄ | Δ |
|---|---|---|---|---|---|
| Antcin A | O | H | H₂ | H | |
| Antcin B | O | H | O | H | |
| Antcin C | O | H | β-OH | H | |
| Antcin D | O | H | O | OH | |
| Antcin E | O | H | H₂ | | 14 |
| Antcin F | O | H | β-OH | | 14 |
| Antcin K | α-OH | OH | β-OH | H | |

In another example of the invention, the compound may be:

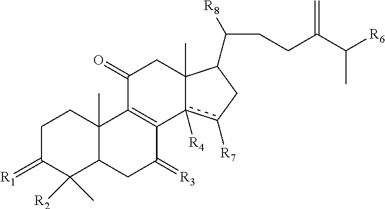

R₇═H; R₈═CH₃

| | R₁ | R₄ | R₅ | R₆ |
|---|---|---|---|---|
| Zhankuic acid B | β-H | H | H | COOH |
| | α-OH | | | |
| Zhankuic acid C | β-H | H | OH | COOH |
| | α-OH | | | |
| Zhankuic acid D | O | H | H | COOEt |
| Zhankuic acid E | β-H | H | OH | COOEt |
| | α-OH | | | |

In one yet example of the invention, the compound may be:

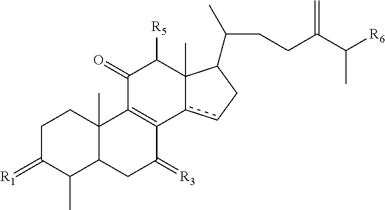

| | R₁ | R₃ | R₅ |
|---|---|---|---|
| methyl antcinate B | O | O | H |
| methyl antcinate G | O | β-OAc | H |
| | | α-H | |
| methyl antcinate H | β-H | O | OH |
| | α-OH | | |
| | O | H₂ | H |

In further example of the invention, the compound may be:

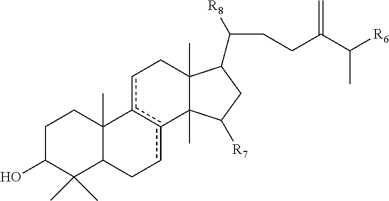

| | R₇ | R₈ | Δ |
|---|---|---|---|
| dehydroeburicoic acid | H | COOH | 7.9 (11) |
| eburicol | H | CH₃ | 8 |
| 15a-acetyl- | OAc | COOH | 7.9 (11) |
| dehydrosulphurenic acid | | | |
| dehydrosulphurenic acid | OH | COOH | 7.9 (11) |
| eburicoic acid | H | COOH | 8 |
| Versisponic acid | OAc | COOH | 8 |
| sulphurenic acid | OH | COOH | 8 |

In one particular example of the invention, the compound may be lanostane:

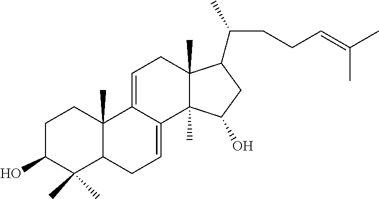

Accordingly, the compound is selected from the group consisting of:

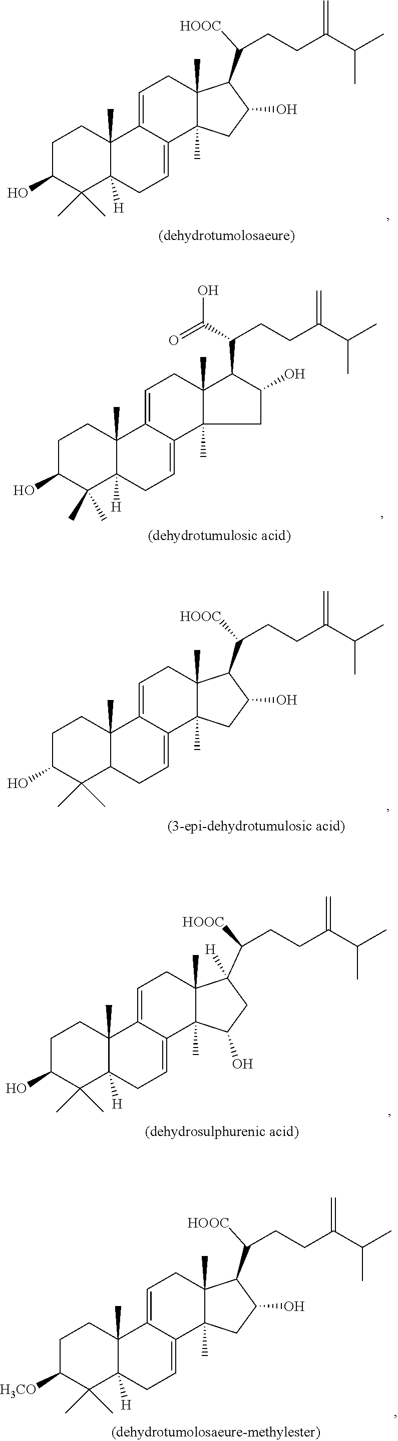

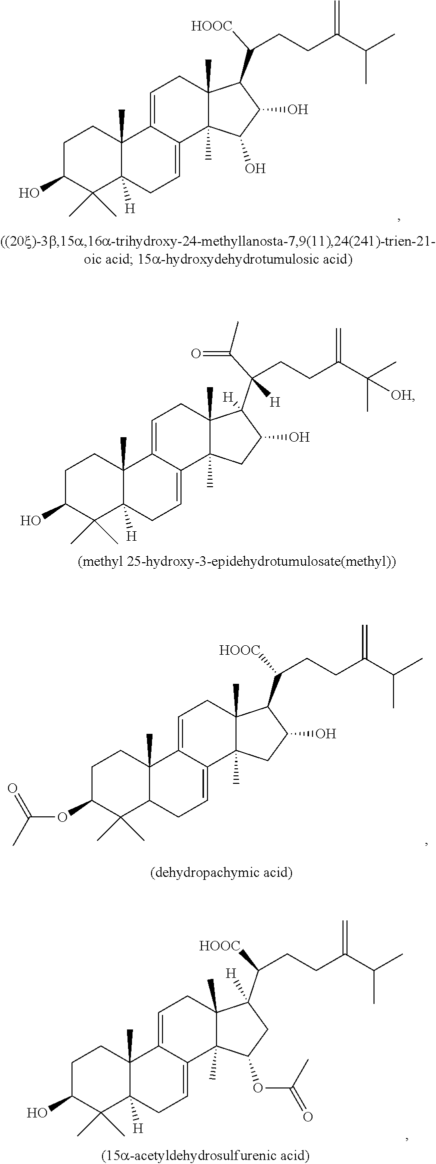

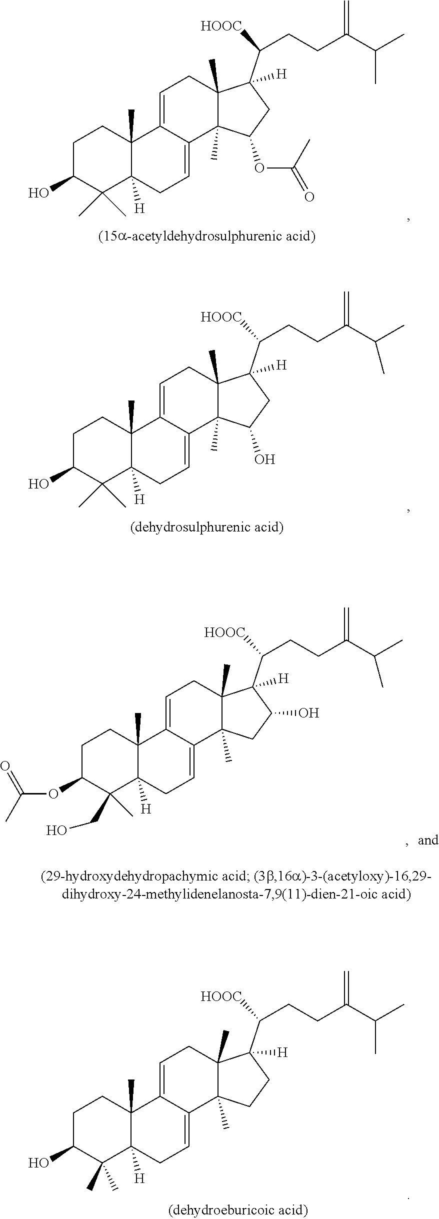

According to the invention, the compound is selected from the group consisting of

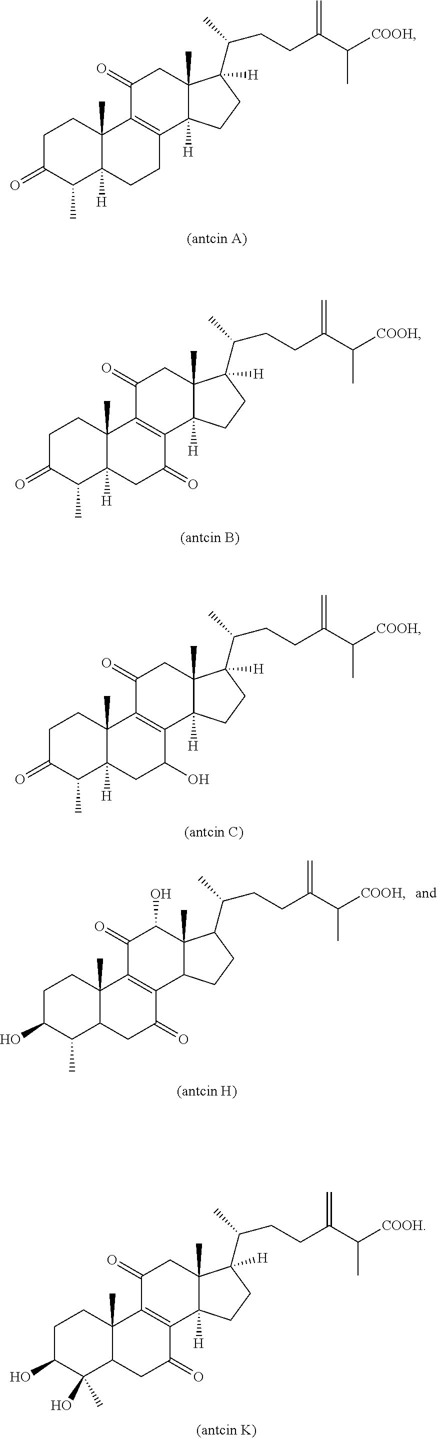 [SMILES: C=C(CC[C@@H](C)[C@H]1CC[C@H]2C3=C(C(=O)C[C@]12C)[C@@]1(C)CCC(=O)[C@@H](C)[C@@H]1CC3)C(C)C(=O)O ; C=C(CC[C@@H](C)[C@H]1CC[C@H]2C3=C(C(=O)C[C@]12C)[C@@]1(C)CCC(=O)[C@@H](C)[C@@H]1CC3=O)C(C)C(=O)O ; C=C(CC[C@@H](C)[C@H]1CC[C@H]2C3=C(C(=O)C[C@]12C)[C@@]1(C)CCC(=O)[C@@H](C)[C@@H]1CC3O)C(C)C(=O)O ; C=C(CC[C@@H](C)C1CCC2C3=C(C(=O)[C@H](O)[C@@]21C)[C@@]1(C)CC[C@H](O)[C@@H](C)C1CC3=O)C(C)C(=O)O ; "and" ; C=C(CC[C@@H](C)[C@H]1CC[C@H]2C3=C(C(=O)C[C@]12C)[C@@]1(C)CC[C@H](O)[C@](C)(O)C1CC3=O)C(C)C(=O)O]

The term “atherosclerotic disease” as used herein refers to atherosclerosis, arteriosclerosis, atheromatous vascular disease, arterial occlusive disease, ischemic stroke, cardiovascular disease, peripheral arterial disease or major organ vascular atherosclerosis, and is characterized by plaque accumulation on vessel walls and vascular inflammation. The plaque is a hallmark of atherosclerotic disease and consists of accumulated intracellular and extracellular lipids, smooth muscle cells, connective tissue, inflammatory cells, and glycosaminoglycans.

The term “preventing,” “prevention” or “prevent” or any other forms of “prevent” used herein refers to an action to block a mechanism or a pathway to a particular event or characteristic or a disease. To stabilize or delay the development or progression of a particular event or characteristic or a disease, or to minimize the chances that a particular event or characteristic will occur.

The term “treating,” “treatment” or “treat” or any other forms of “treat” as used herein refers to any and all uses which remedy a disease state or symptoms, or otherwise prevent, hinder, retard, or reverse the progression of disease or other undesirable symptoms in any way whatsoever.

The term “inhibiting,” “inhibition” or “inhibit” or any other forms of “inhibit” used herein refers to an action of reduction or stop features of an event or characteristic (e.g. atherosclerosis) ora disease.

The term “reducing,” “reduction” or “reduce” or any other forms of “reduce” used herein refers to an action of lowering or an event or characteristic (e.g. atherosclerosis). It is understood that this is typically in relation to some standard expected value, in other word it is relative, but that is not always necessary for the standard or relative value to be referred to.

The term “reversing,” “reversal” or “reverse” or any other forms of “reverse” used herein refers to an action to recover a patient suffering from a disease to her/his initial healthy state.

The term “subject” as used herein includes human or non-human animals, such as companion animals (e.g. dogs, cats, etc.), farm animals (e.g. cattle, sheep, pigs, horses, etc.), or experimental animals (e.g. rats, mice, guinea pigs, etc.).

The term “therapeutically effective amount” as used herein refers to an amount of a pharmaceutical agent which, as compared to a corresponding subject who has not received such amount, results in an effect in treatment, healing, prevention, or amelioration of a disease, disorder, or side effect, or a decrease in the rate of advancement of a disease or disorder. The term also includes within its scope amounts effective to enhance normal physiological function.

For use in therapy, the therapeutically effective amount of the compound is formulated as a pharmaceutical composition for administration. Accordingly, the invention further provides a pharmaceutical composition comprising a therapeutically effective amount of the preparation of Antrodia camphorata or the active compounds isolated from Antrodia camphorata, and one or more pharmaceutically acceptable carriers.

For the purpose of delivery and absorption, a therapeutically effective amount of the active ingredient according to the present invention may be formulated into a pharmaceutical composition in a suitable form with a pharmaceutically acceptable carrier. Based on the routes of administration, the pharmaceutical composition of the present invention comprises preferably from 0.1% to 100% in weight of the total weight of the active ingredient.

The term “pharmaceutically acceptable carrier” used herein refers to a carrier(s), diluent(s) or excipient(s) that is acceptable, in the sense of being compatible with the other ingredients of the formulation and not deleterious to the subject to be administered with the pharmaceutical composition. Any carrier, diluent or excipient commonly known or used in the field may be used in the invention, depending to the requirements of the pharmaceutical formulation. According to the invention, the pharmaceutical composition may be adapted for administration by any appropriate route, including but not limited to oral, rectal, nasal, topical, vaginal, or parenteral route. In one particular example of the invention, the pharmaceutical composition is formulated for oral administration. Such formulations may be prepared by any method known in the art of pharmacy.

As used herein, “pharmaceutically acceptable” means that the carrier is compatible with the active ingredient in the composition, and preferably can stabilize said active ingredient and is safe to the individual receiving the treatment. Said carrier may be a diluent, vehicle, excipient, or matrix to the active ingredient. Some examples of appropriate excipients include lactose, dextrose, sucrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates; sweeteners; and flavoring agents. The composition of the present invention can provide the effect of rapid, continued, or delayed release of the active ingredient after administration to the patient.

According to the present invention, the form of said composition may be tablets, pills, powder, lozenges, packets, troches, elixers, suspensions, lotions, solutions, syrups, soft and hard gelatin capsules, suppositories, sterilized injection fluid, and packaged powder.

The composition of the present invention may be delivered via any physiologically acceptable route, such as oral, parenteral (such as intramuscular, intravenous, subcutaneous, and intraperitoneal), transdermal, suppository, and intranasal methods. Regarding parenteral administration, it is preferably used in the form of a sterile water solution, which may comprise other substances, such as salts or glucose sufficient to make the solution isotonic to blood. The water solution may be appropriately buffered (preferably with a pH value of 3 to 9) as needed. Preparation of an appropriate parenteral composition under sterile conditions may be accomplished with standard pharmacological techniques well known to persons skilled in the art.

According to the present invention, the composition/pharmaceutical composition described herein may also be administered to a human or a non-human animal in a dietary supplement for decreasing the concentration of LDL-cholesterol and increasing the concentration of HDL-cholesterol in the blood to reduce the risk of atherosclerosis and vascular disease. Dietary supplements incorporating the composition/pharmaceutical composition can be prepared by adding daidzein to a food in the process of preparing the food, independent of the source of the composition/pharmaceutical composition. The foods to which the composition/pharmaceutical composition may be added include almost all foods. For example, the composition/pharmaceutical composition can be added to foods including, but not limited to, meats such as ground meats, emulsified meats, marinated meats, and meats injected with daidzein; beverages such as nutritional beverages, sports beverages, protein fortified beverages, juices, milk, milk alternatives, and weight loss beverages; cheeses such as hard and soft cheeses, cream cheese, and cottage cheese; frozen desserts such as ice cream, ice milk, low fat frozen desserts, and non-diary frozen desserts; yogurts; soups; puddings; bakery products; salad dressings; and dips and spreads such as mayonnaise and chip dips. The composition/pharmaceutical composition is added to the food in an amount selected to deliver a desired dose of the composition/pharmaceutical composition to the consumer of the food.

According to the present invention, the method and composition/pharmaceutical composition described herein could be administrated a subject in combination with at least one additional atherosclerosis therapeutic agent. Exemplified atherosclerosis therapeutic agents which are responsive include, without limitation, statins, fibrates, niacin, Ezetimibe, bile acid sequestrants (such as cholestyramine, colestipol or colesevelam), alirocumab, evolocumab, aspirin, clopidogrel, ticagrelor, prasugrel, and warfarin.

The present invention is further illustrated by the following examples, which are provided for the purpose of demonstration rather than limitation.

### EXAMPLES

Materials and Methods

1. Preparation of an Extract of Antrodia camphorata and its Active Fraction

100 grams of Antrodia camphorata fruiting body was heat-recirculated with methanol for 6 hours, and the extract was collected and dried under reduced pressure to obtain 15 grams of the Antrodia camphorate methanol extract.

15 grams of the Antrodia camphorata methanol extract as obtained above was taken, filled with silicon dioxide, and subjected to a gradient elution with the eluant “hexane/ethyl acetate/methanol” in a column separation (3×12 cm) to obtain active fractions, including ARH003 and ARH004. Pure compound derived from Antrodia camphorata fruiting body extracts was further isolated.

2. Experimental Model

2 to 3 kg male, New Zealand White rabbits were individually caged and housed in temperature and humidity-controlled rooms. Light-dark cycles were 12 h each. After several days of acclimation, the animals were sequentially assigned to six feeding groups: standard rabbit chow (ND), standard rabbit chow containing 0.5% cholesterol (HF), standard rabbit chow containing both 0.5% cholesterol and 10 mg/kg Lovastatin (L), standard rabbit chow containing both 0.5% cholesterol and 1% ARH003 (ARH003), standard rabbit chow containing both 0.5% cholesterol and 1% ARH004 (ARH004), standard rabbit chow containing both 0.5% cholesterol and 10 mg/kg Pure compound (P).

Except standard rabbit chow, others groups were given standard rabbit chow containing 0.5% cholesterol for 4 weeks. The daily feeding amount for each rabbit was 50 g/kg body weight per day.

Diets were administered for 8 weeks, after the animals had adjusted to their new environment. At the beginning and end of the 12 weeks study, the rabbits were anesthetized by an intramuscular injection of Zoletil 50 (1 mL/kg) (Virbac Ltd., France), and blood samples were harvested. Finally, the aortas (from aortic arch to the bifurcation of the iliac arteries) and whole livers were collected from the rabbits after they were sacrificed for further histopathological analyses.

3. Blood Chemistry Analysis

The animals were fasted overnight before blood drawing. The blood was collected from the marginal ear veins of rabbits into BD Vacutainer EDTA Blood Collection Tubes. Plasma was separated by centrifugation at 3,000 rpm at 4.0 for 10 min. Measurements for changes in blood chemistry parameters included serum levels of low-density lipoprotein (LDL), cholesterol (Chol), triglycerides (TG), glutamate oxaloacetate transaminase (GOT), and glutamate pyruvate transaminase (GPT).

4. Aortic Fatty Streak Staining method

The aortas were opened longitudinally to expose the intimal surface and rinsed gently with normal saline. Aortas were incubated in 2% (w/v) Sudan IV, rinsed with several concentrations (100%, 90%, 80%, 70%, and 60%) of ethanol for 1 min, and then rinsed with pure water. The photographs were acquired using a digital camera (Nikon D80, Japan) and quantified on an Alpha Imager 2200 documentation system (Alpha Innotech, USA).

5. Histopathological Examination

Conventional techniques of paraffin embedded tissues sectioning and haematoxylin-eosin (HE) staining were used in this study. Fresh specimens were cut and fixed in either alcohol or an aldehyde-based fixative. Following fixation, the tissue specimens were rinsed in H₂O. Then the tissue specimens were stained by Mayer's haematoxylin and 1% eosin Y and examined under a bright-field microscope.

6. Cryosectioning of Liver Tissues

The rabbit liver tissues were perfused with normal saline and fixed in 10% (v/v) formalin-neutralized solution (J. T. Baker, Inc., USA) for 24 hr. Afterward, the tissues were embedded in Tissue Tek OCT Compound (#4583; Sakura Finetek Inc., USA). Embedded tissues were cut into 10 μm thick slices and stained with Sudan IV and hematoxylin (Merck, USA). Briefly, the slices were washed with pure water for 1 min to remove the OCT compound, washed with 50% (v/v) ethanol for 30 sec, and then stained with 2% (w/v) Sudan IV for 1 hr. After further washing with 50% (v/v) ethanol and pure water for 2 min, the slices were counterstained with hematoxylin. Photographs were acquired using a microscope equipped with a 10-fold magnification objective and quantified on an Alpha Imager 2200 documentation system (Alpha Innotech, USA). The manifestation of fatty liver progression was presented as the percentage of the area of oil droplets to the total liver tissues (cells).

7. Statistical Analysis

All values are expressed as mean±SE. Each value is the mean of at least three experiments in each drug in vitro experiments. Student's t-test is used for statistical comparison. * indicates that the values are significantly different from the control (*, p<0.05; **, P<0.01).

### Example 1

Inhibitory Effect of Antrodia camphorata Fruit Body Extracts or the Derived Pure Compound on Serum Triglyceride and Total Cholesterol Levels

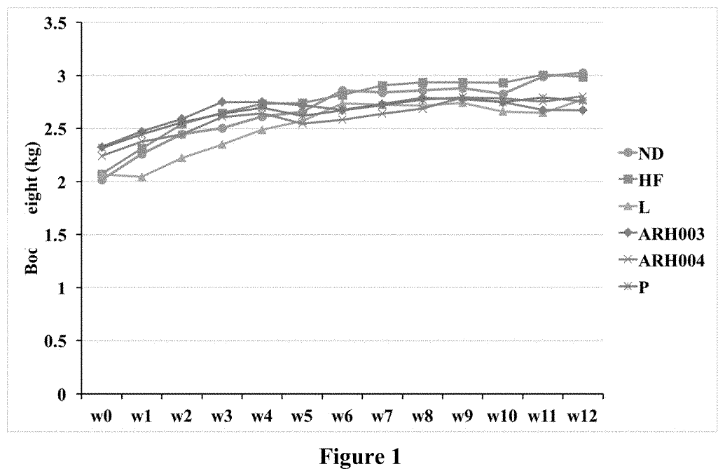

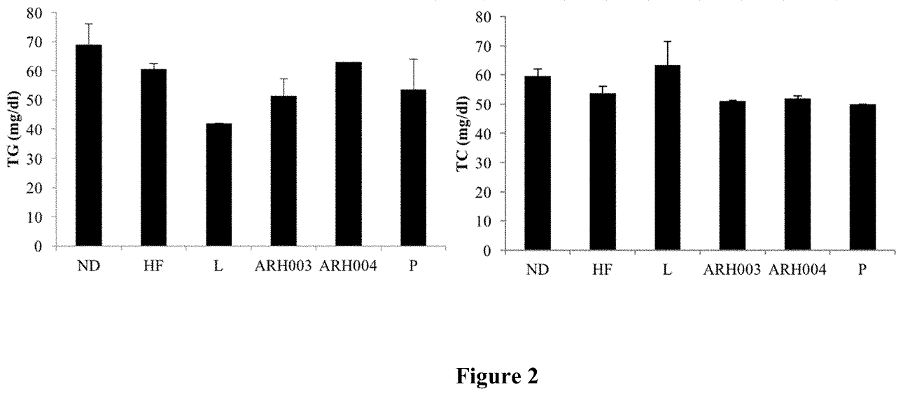

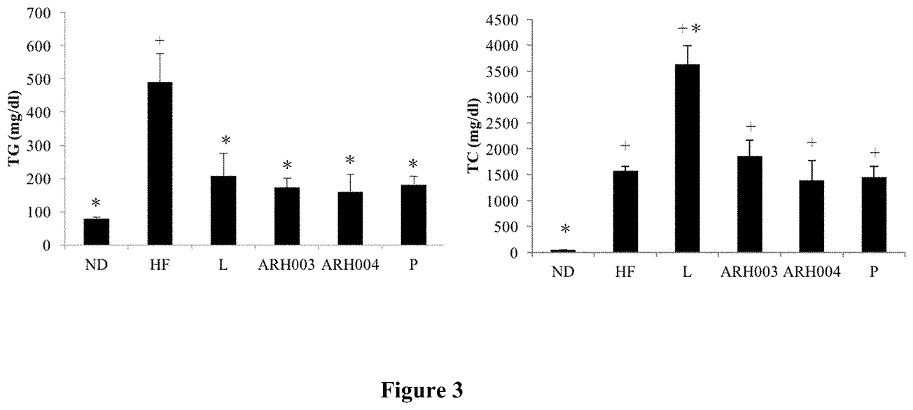

Supplementing the high fat diet of the rabbits alone or with lovastatin, Antrodia camphorata fruit body extracts, or the derived pure compound did not affect the body weight gains during the intervention (FIG. 1). Furthermore, at the beginning of the treatment period, levels of triglyceride and total cholesterol among groups did not exhibit significant changes (FIG. 2). However, supplementing the high fat diet caused a significant increase in the serum triglyceride and total cholesterol levels, but the rabbits treated with Antrodia camphorata fruit body extracts, or the derived pure compound exhibited a significant improvement in their serum triglyceride and total cholesterol levels (FIG. 3). Of noted, the lovastatin group rabbits exhibited a higher serum total cholesterol levels compared to that of high fat diet group animals.

### Example 2

Inhibitory Effect of Antrodia camphorata Fruit Body Extracts or the Derived Pure Compound on the Formation of Fatty Steak

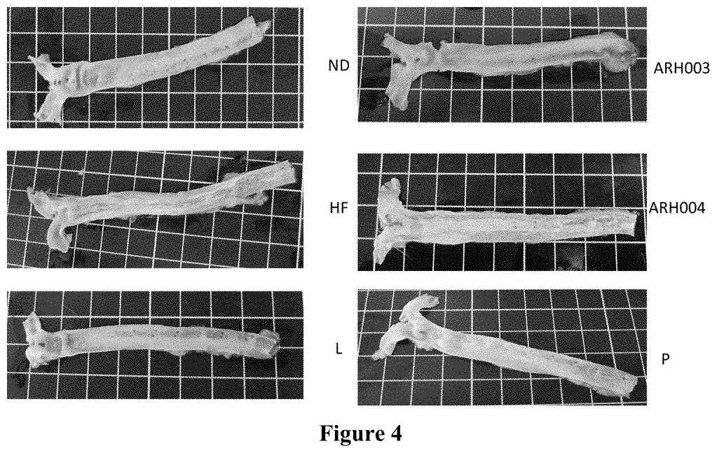

The earliest visible lesion of atherosclerosis is the fatty streak, and with time the fatty streak evolves into a fibrous plaque, the hallmark of established atherosclerosis. Accordingly, the study on fatty streak formation revealed that Antrodia camphorata fruit body extracts, or the derived pure compound significantly reduced the atherosclerotic lesions as shown in FIG. 4.

### Example 3

Protective Effect of Antrodia camphorata Fruit Body Extracts or the Derived Pure Compound on the Neointimal Formation

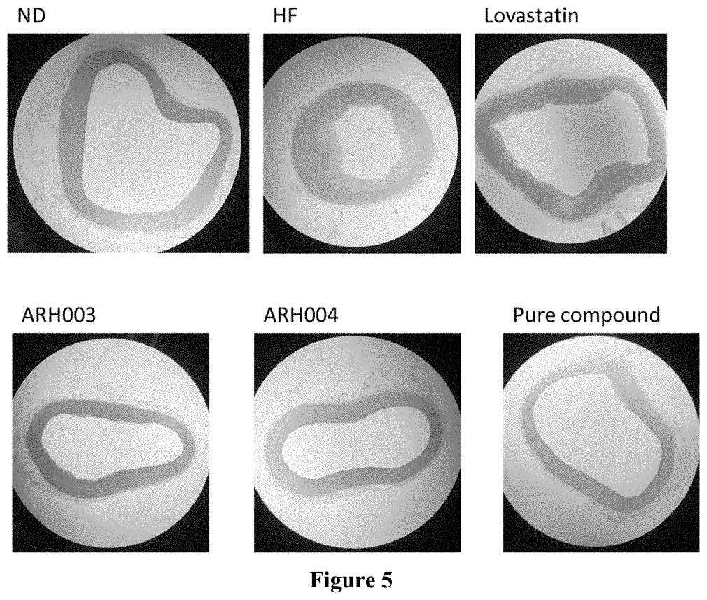

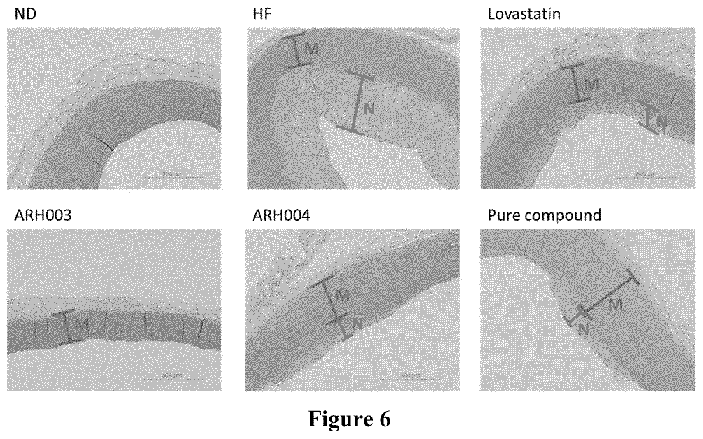

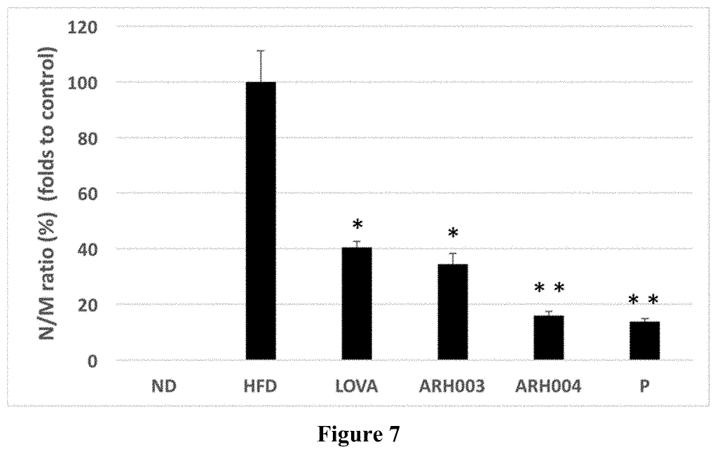

The histologic features of coronary artery of high fat diet group rabbits showed that the intimal layer becomes thicker and the diameter of the luminalis smaller due to the atherosclerotic plaques. The features of coronary artery of Antrodia camphorata fruit body extracts, or the derived pure compound groups rabbits showed almost normal artery without plaque (FIGS. 5 and 6). Furthermore, Antrodia camphorata fruit body extracts, or the derived pure compound groups rabbits displayed a >60% reduction in neointima-to-media ratio manifesting the vascular restenosis (FIG. 7).

### Example 4

Protective Affect of Antrodia camphorata Fruit Body Extracts or the Derived Pure Compound on Hepatic Lipid Accumulation

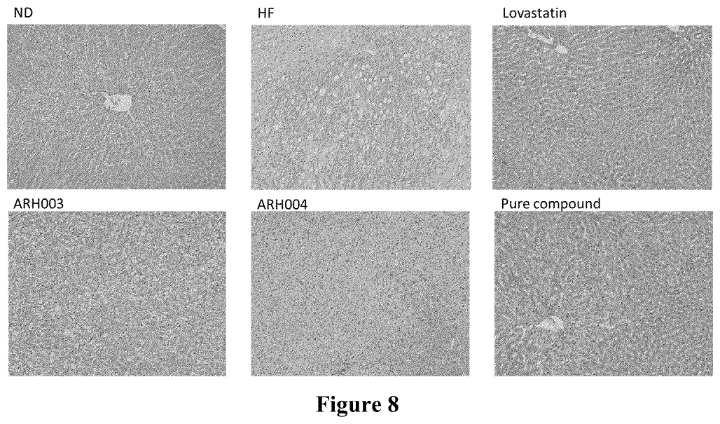

As mentioned above, high fat diet-fed rabbits exhibited increased serum triglyceride and total cholesterol levels. The similar profile was further demonstrated by histopathological analysis with high-fat-diet-induced hepatic lipid accumulation, while this phenomenon was diminished by the treatment of Antrodia camphorata fruit body extracts or the derived pure compound (FIG. 8).

Taken together, Antrodia camphorata fruit body extracts or the derived pure compound disclosed herein provide protective effects on atherosclerotic plaque formation and hepatic lipid accumulation, and would be beneficial in treating atherosclerotic disease.

The above description merely relates to preferred embodiments in the present invention, and it should be pointed out that, for a person of ordinary skill in the art, some improvements and modifications can also be made under the premise of not departing from the principle of the present invention, and these improvements and modifications should also be considered to be within the scope of protection of the present invention.

1. A method for inhibiting, reducing or reversing atherosclerosis comprising
administering to a subject in need thereof an effective amount of a composition or pharmaceutical composition containing a preparation of Antrodia camphorata, and/or one or more active compounds isolated from Antrodia camphorata.

2. A method for treating or preventing an atherosclerotic disease comprising
administering to a subject in need thereof an effective amount of a composition or pharmaceutical composition containing a preparation of Antrodia camphorata, and/or one or more active compounds isolated from Antrodia camphorata.

3. The method of claim 2, wherein the atherosclerotic disease is selected from atherosclerosis, arteriosclerosis, atheromatous vascular disease, arterial occlusive disease, ischemic stroke, cardiovascular disease, peripheral arterial disease or major organ vascular atherosclerosis, and is characterized by plaque accumulation on vessel walls and vascular inflammation.

4. The method of claim 1, wherein the preparation of Antrodia camphorate is one or more selected from the group consisting of an extract of Antrodia camphorate, an extract of a dish culture of Antrodia camphorate, an extract of Antrodia camphorata fruit body, and active compounds isolated from the above-mentioned extracts, and the derivatives thereof.

5. The method of claim 1, wherein the active compound isolated from Antrodia camphorata is one or more selected from the group consisting of the following: 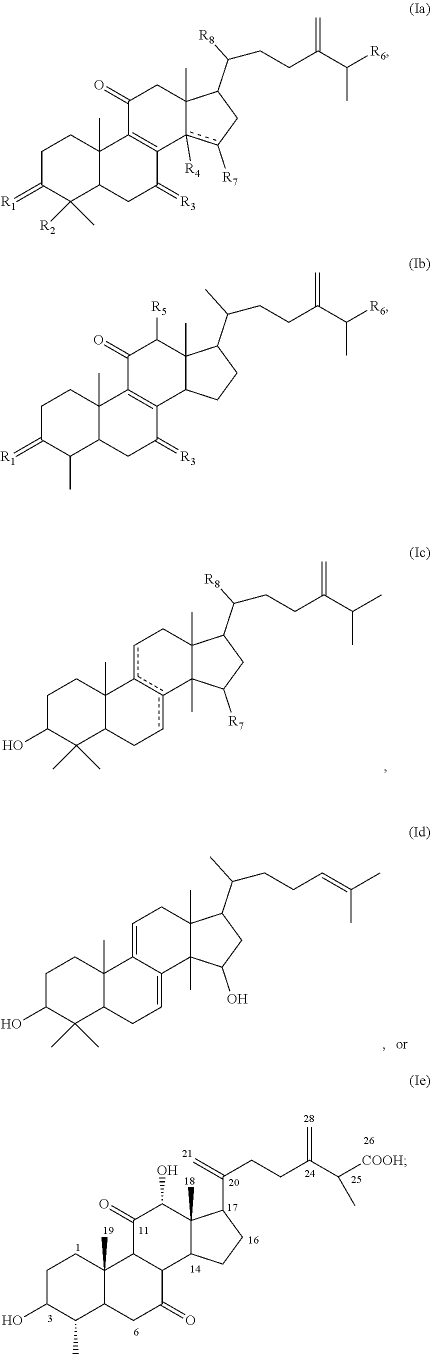
wherein R₁ is O, α-OH or β-H; R₂ is H or OH; R₃ is O, α-H, β-OAc or H₂; R₄ is H or OH; R₅ is H or OH; R₆ is COOH or COO(CH₂)n-CH₃; R₇ is H, OH, or OAc; R₈ is CH₃ or COOH; the dotted line represents a single bond or a double bond; n is an integer from 0-3.

6. The method of claim 1, wherein the active compound isolated from Antrodia camphorata is:

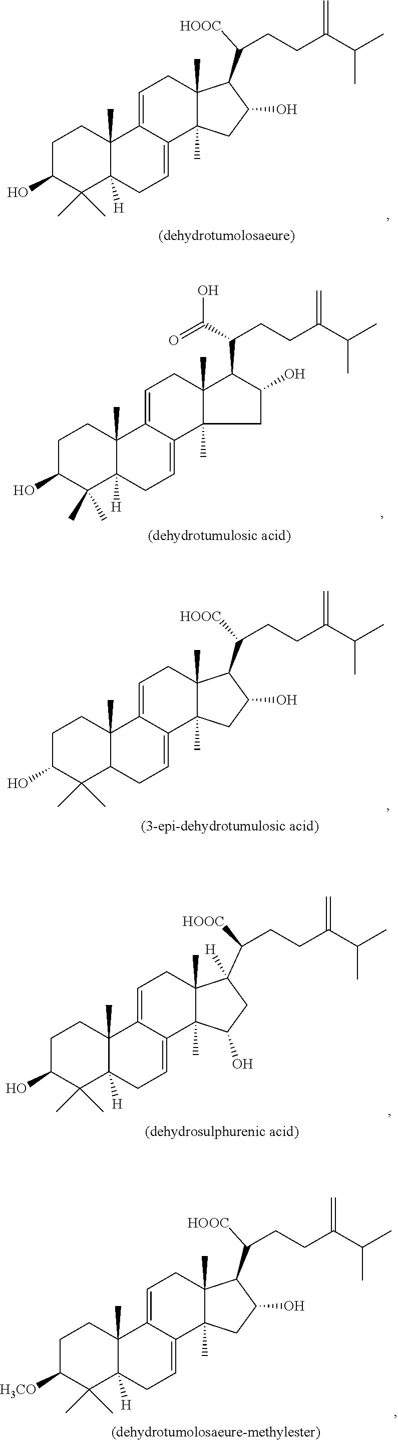

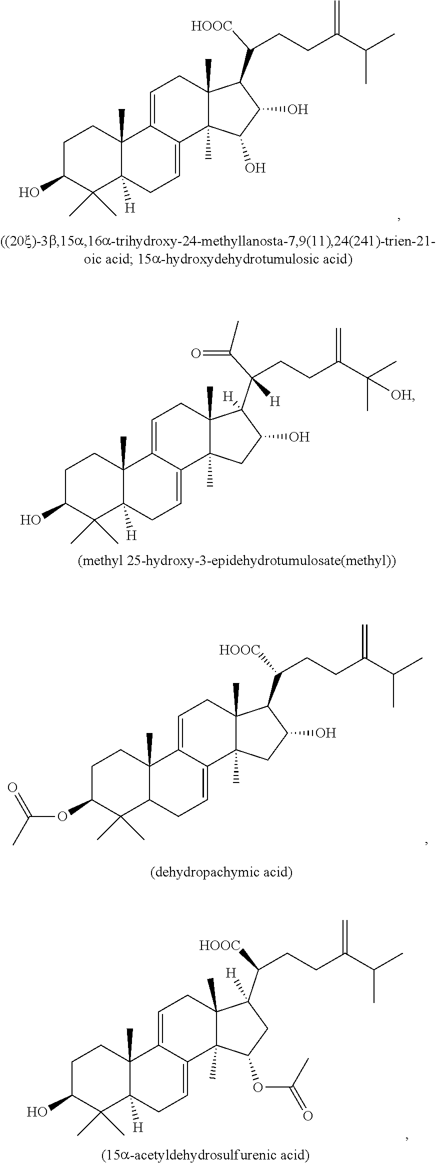

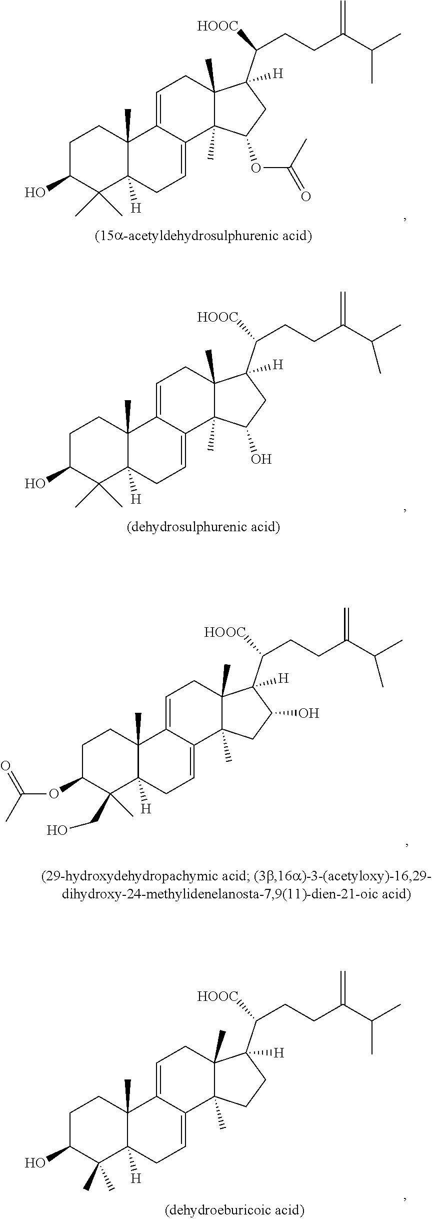

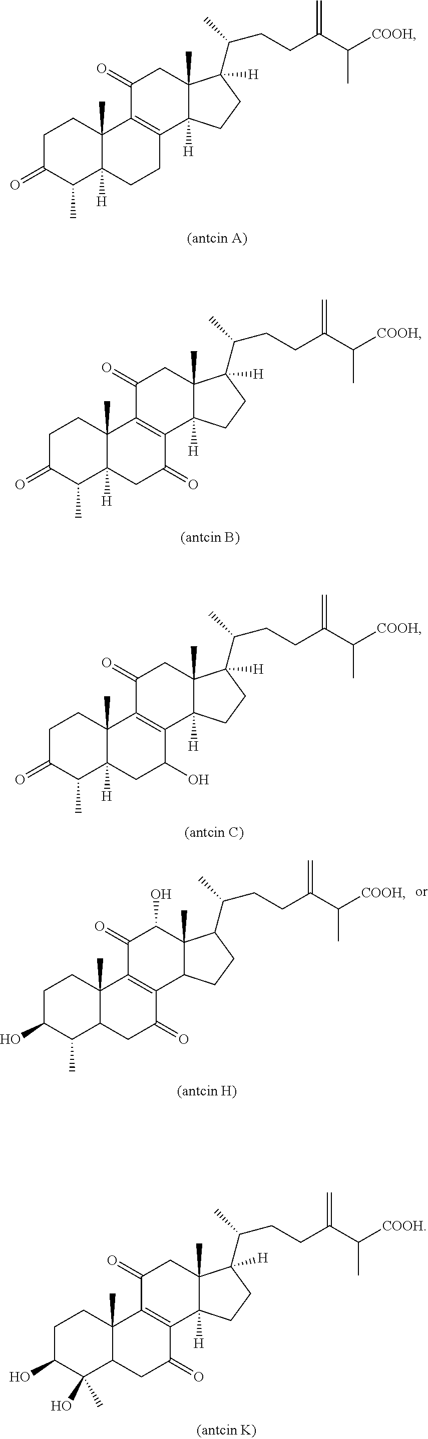 [SMILES: C=C(CC[C@@H](C)[C@H]1CC[C@H]2C3=C(C(=O)C[C@]12C)[C@@]1(C)CCC(=O)[C@@H](C)[C@@H]1CC3)C(C)C(=O)O ; C=C(CC[C@@H](C)[C@H]1CC[C@H]2C3=C(C(=O)C[C@]12C)[C@@]1(C)CCC(=O)[C@@H](C)[C@@H]1CC3=O)C(C)C(=O)O ; C=C(CC[C@@H](C)[C@H]1CC[C@H]2C3=C(C(=O)C[C@]12C)[C@@]1(C)CCC(=O)[C@@H](C)[C@@H]1CC3O)C(C)C(=O)O ; C=C(CC[C@@H](C)C1CCC2C3=C(C(=O)[C@H](O)[C@@]21C)[C@@]1(C)CC[C@H](O)[C@@H](C)C1CC3=O)C(C)C(=O)O ; "or" ; C=C(CC[C@@H](C)[C@H]1CC[C@H]2C3=C(C(=O)C[C@]12C)[C@@]1(C)CC[C@H](O)[C@](C)(O)C1CC3=O)C(C)C(=O)O]

7. A composition for preventing or treating an atherosclerotic disease comprising
a therapeutically effective amount of a composition or pharmaceutical composition containing a preparation of Antrodia camphorata, and/or one or more active compounds isolated from Antrodia camphorata.

8. The composition of claim 7, comprising one or more therapeutically acceptable carrier.

9. The composition of claim 7, further comprising at least one additional therapeutic agent.

10. The composition of claim 7, wherein the atherosclerotic disease is selected from atherosclerosis, arteriosclerosis, atheromatous vascular disease, arterial occlusive disease, ischemic stroke, cardiovascular disease, peripheral arterial disease or major organ vascular atherosclerosis, and is characterized by plaque accumulation on vessel walls and vascular inflammation.

11-13. (canceled)

14. The method of claim 2, wherein the preparation of Antrodia camphorate is one or more selected from the group consisting of an extract of Antrodia camphorate, an extract of a dish culture of Antrodia camphorate, an extract of Antrodia camphorata fruit body, and active compounds isolated from the above-mentioned extracts, and the derivatives thereof.

15. The method of claim 2, wherein the active compound isolated from Antrodia camphorata is one or more selected from the group consisting of the following: 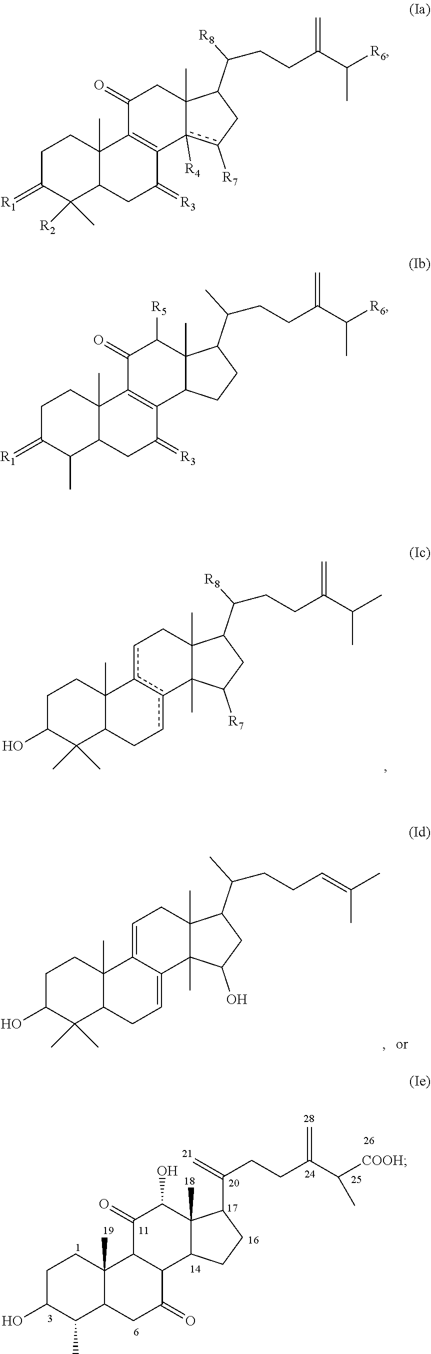
wherein R₁ is O, α-OH or β-H; R₂ is H or OH; R₃ is O, α-H, β-OAc or H₂; R₄ is H or OH; R₅ is H or OH; R₆ is COOH or COO(CH₂)n-CH₃; R₇ is H, OH, or OAc; R₈ is CH₃ or COOH; the dotted line represents a single bond or a double bond; n is an integer from 0-3.

16. The method of claim 2, wherein the active compound isolated from Antrodia camphorata is:

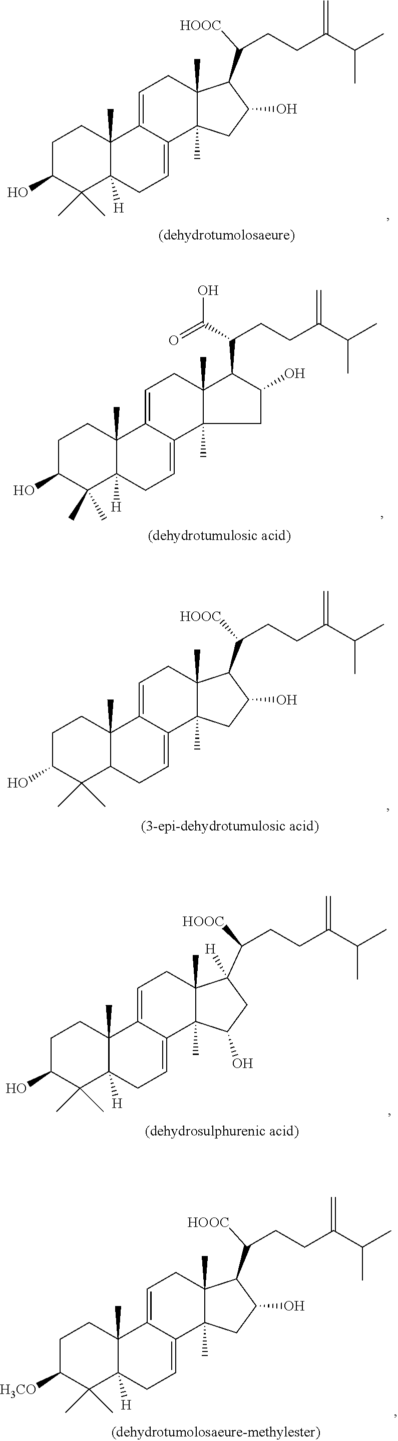

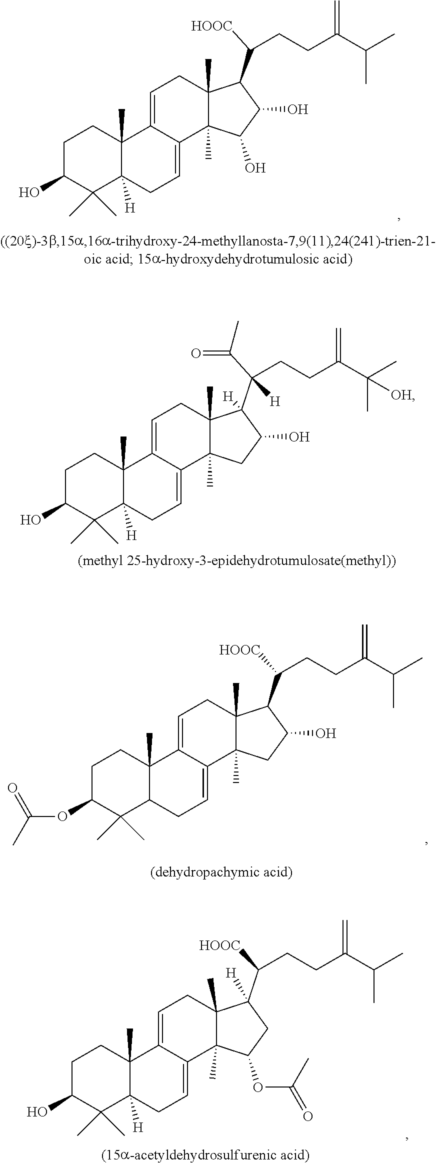

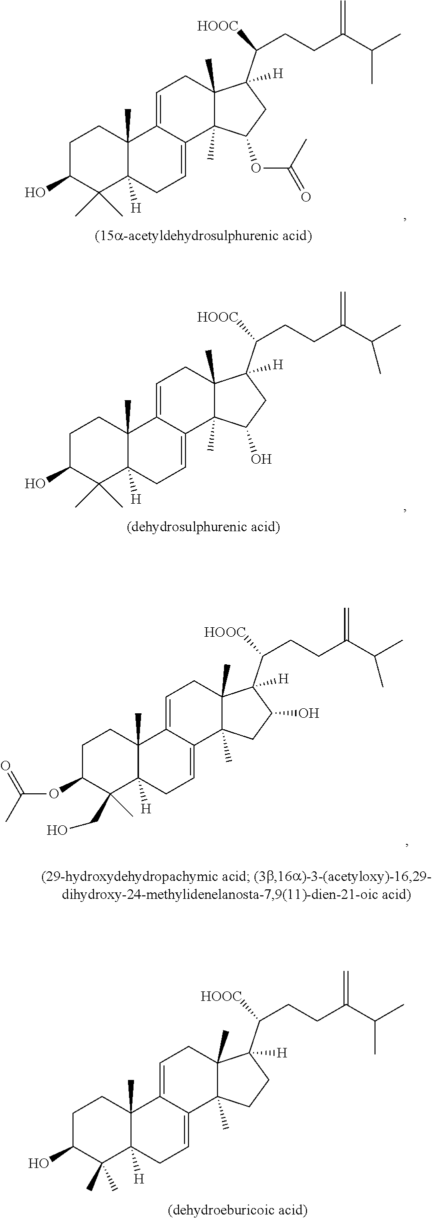

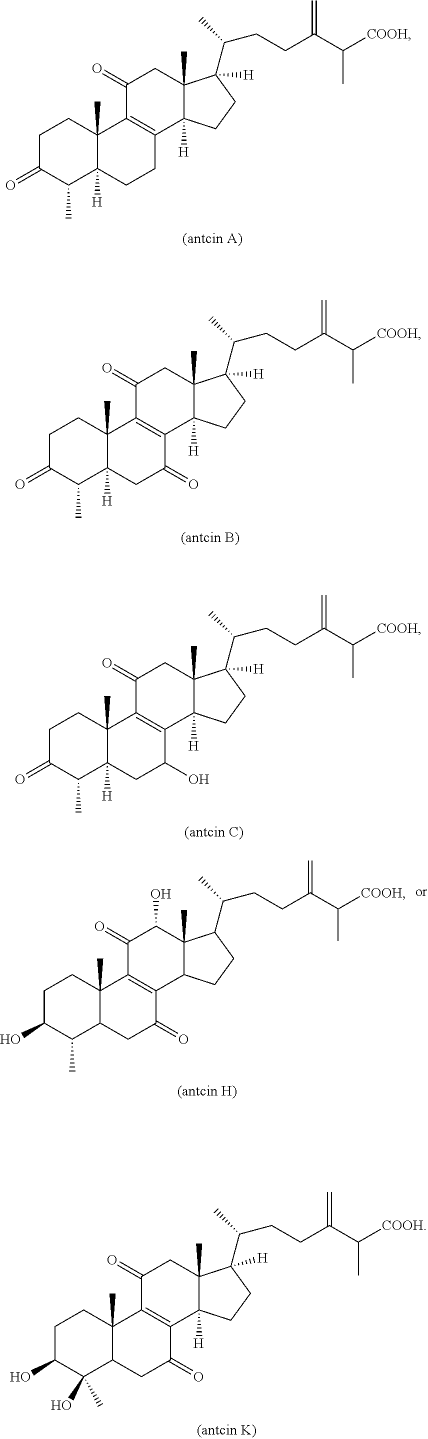 [SMILES: C=C(CC[C@@H](C)[C@H]1CC[C@H]2C3=C(C(=O)C[C@]12C)[C@@]1(C)CCC(=O)[C@@H](C)[C@@H]1CC3)C(C)C(=O)O ; C=C(CC[C@@H](C)[C@H]1CC[C@H]2C3=C(C(=O)C[C@]12C)[C@@]1(C)CCC(=O)[C@@H](C)[C@@H]1CC3=O)C(C)C(=O)O ; C=C(CC[C@@H](C)[C@H]1CC[C@H]2C3=C(C(=O)C[C@]12C)[C@@]1(C)CCC(=O)[C@@H](C)[C@@H]1CC3O)C(C)C(=O)O ; C=C(CC[C@@H](C)C1CCC2C3=C(C(=O)[C@H](O)[C@@]21C)[C@@]1(C)CC[C@H](O)[C@@H](C)C1CC3=O)C(C)C(=O)O ; "or" ; C=C(CC[C@@H](C)[C@H]1CC[C@H]2C3=C(C(=O)C[C@]12C)[C@@]1(C)CC[C@H](O)[C@](C)(O)C1CC3=O)C(C)C(=O)O]

17. The composition according to claim 7, wherein the therapeutically effective amount of the preparation of Antrodia camphorata is one or more selected from the group consisting of an extract of Antrodia camphorate, an extract of a dish culture of Antrodia camphorate, an extract of Antrodia camphorata fruit body, and active compounds isolated from the above-mentioned extracts, and the derivatives thereof.

18. The composition of claim 7, wherein the active compound isolated from Antrodia camphorata is one or more selected from the group consisting of the following: 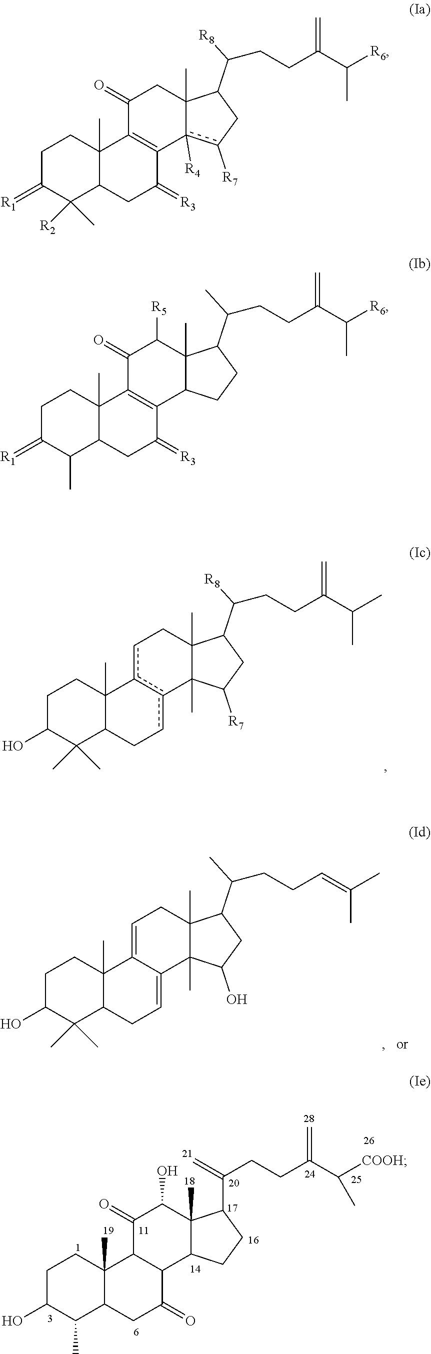
wherein R₁ is O, α-OH or β-H; R₂ is H or OH; R₃ is O, α-H, β-OAc or H₂; R₄ is H or OH; R₅ is H or OH; R₆ is COOH or COO(CH₂)n-CH₃; R₇ is H, OH, or OAc; R₈ is CH₃ or COOH; the dotted line represents a single bond or a double bond; n is an integer from 0-3.

19. The composition of claim 17, wherein the active compound isolated from Antrodia camphorata is: 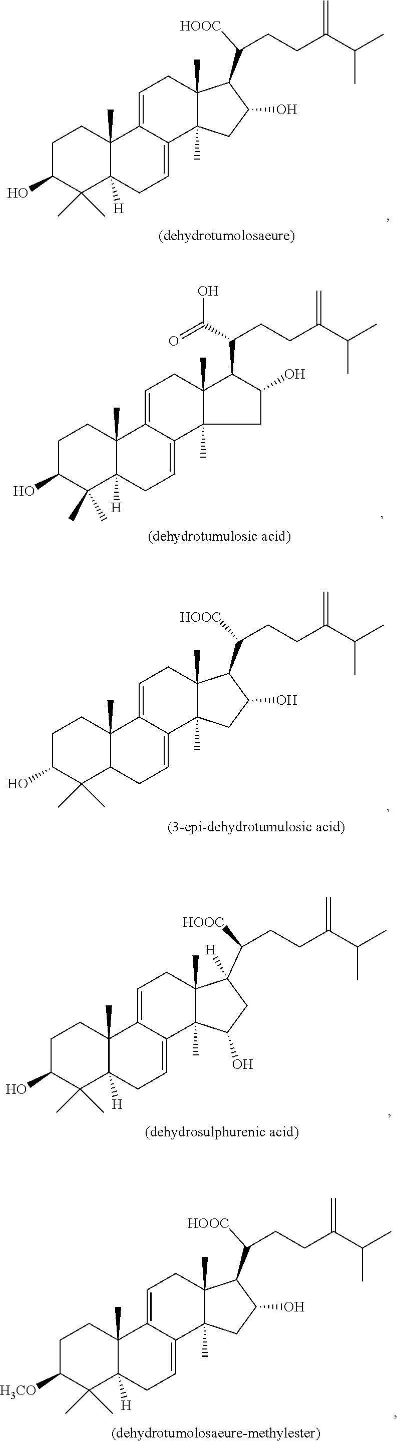 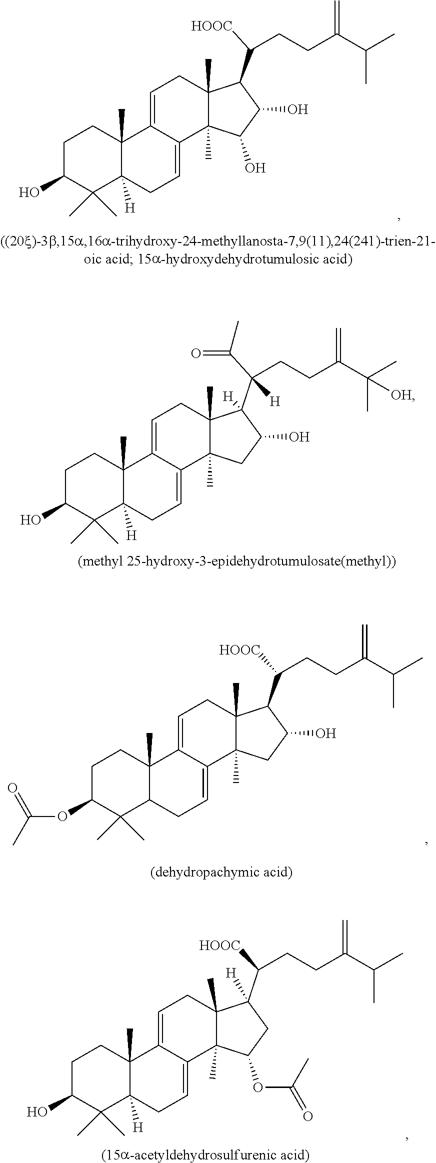 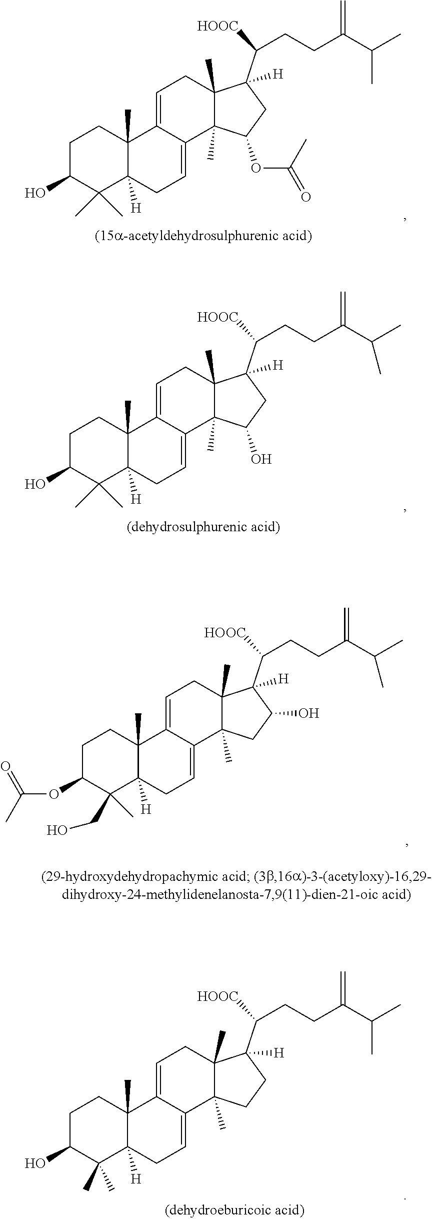 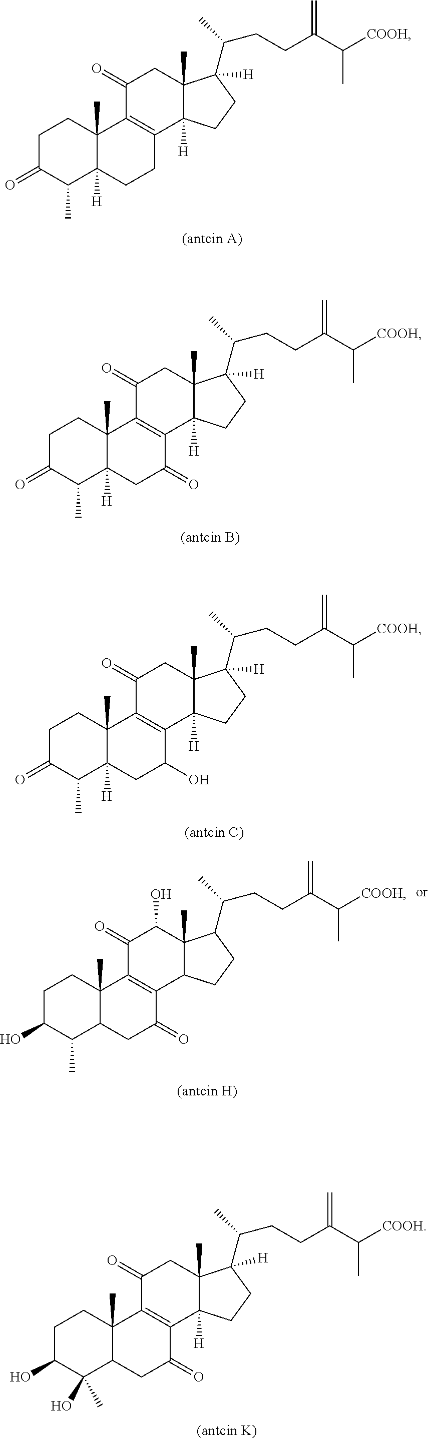 [SMILES: C=C(CC[C@@H](C)[C@H]1CC[C@H]2C3=C(C(=O)C[C@]12C)[C@@]1(C)CCC(=O)[C@@H](C)[C@@H]1CC3)C(C)C(=O)O ; C=C(CC[C@@H](C)[C@H]1CC[C@H]2C3=C(C(=O)C[C@]12C)[C@@]1(C)CCC(=O)[C@@H](C)[C@@H]1CC3=O)C(C)C(=O)O ; C=C(CC[C@@H](C)[C@H]1CC[C@H]2C3=C(C(=O)C[C@]12C)[C@@]1(C)CCC(=O)[C@@H](C)[C@@H]1CC3O)C(C)C(=O)O ; C=C(CC[C@@H](C)C1CCC2C3=C(C(=O)[C@H](O)[C@@]21C)[C@@]1(C)CC[C@H](O)[C@@H](C)C1CC3=O)C(C)C(=O)O ; "or" ; C=C(CC[C@@H](C)[C@H]1CC[C@H]2C3=C(C(=O)C[C@]12C)[C@@]1(C)CC[C@H](O)[C@](C)(O)C1CC3=O)C(C)C(=O)O]
